(19) United States
(12) Patent Application Publication
(10) Pub. No.: US 2010/0037713 A1
(43) Pub. Date: Feb. 18, 2010

(54) Pharmaceutical Analysis Apparatus and Method

(76) Inventor: Vijay Mohan Iyer, Mississauga (CA)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/530,286

(22) PCT Filed: Mar. 6, 2008
(86) PCT No.: PCT/US08/55997

§ 371 Date: Sep. 8, 2009

Related U.S. Application Data
(60) Provisional application No. 60/893,650, filed on Mar. 8, 2007.

(51) Int. Cl. G01N 33/15; G01N 21/01

(52) U.S. Cl. 73/865.6; 73/866; 250/428

(57) ABSTRACT

An apparatus and method are provided for analyzing the release of active agent(s) from pharmaceutical and pharmaceutical-like products. The apparatus and method provide for more accurate simulation of the conditions in the GI tract. A sinker is utilized to hold the dosage form so that substantially all of the surfaces of the dosage form are equally agitated by the dissolution medium.

### FIELD OF THE INVENTION

The present invention relates to the analysis of pharmaceutical and pharmaceutical-like products. More particularly, the present invention relates to an apparatus and process for analyzing and/or predicting the release of active agents in pharmaceutical and pharmaceutical-like products.

### DESCRIPTION OF RELATED ART

Conventional dissolution devices include a basket-type, a paddle-type, a reciprocating cylinder-type, and a laminar flow-through column-type. For example, the traditional paddle-type dissolution device has a glass, round-bottomed vessel with an impeller for mixing the contents of the vessel. The paddle-type device can also have an auto-sampler tube inserted into the vessel to collect samples at selected times from an aqueous solution in the vessel. A dosage form to be analyzed is dropped into the vessel and falls to the bottom, where it will remain during the dissolution run. The basket and reciprocating cylinder-type dissolution devices similarly provide for mixing of the solution in the device while the tablet rests in the basket/cylinder. The flow-through column-type dissolution device has a laminar flowing liquid medium into which the tablet is introduced and in which it dissolves over time. The liquid exiting the column is analyzed for the dissolved active agent.

These conventional dissolution devices suffer from distinct drawbacks in gathering dissolution data. The degree of agitation in rotating baskets far exceeds what might be expected in vivo. The wire basket can clog due to adhering substances, which can result in poor reproducibility. Additionally, particles of the dosage form can fall from the rotating basket and sink to the bottom of the flask where they will not be subjected to the same degree of agitation present inside the basket. Matrix tablets and tablets containing polymeric materials are particularly prone to clogging fine-mesh baskets, leading to slower dissolution and erroneous results.

In paddle-type devices, matrix tablets tend to stick to the sidewall of the round-bottom vessels, resulting in a reduction of the exposed surface area to the dissolution media. The inconsistent orientation of the dosage form at the bottom of the vessel can give variable data. These devices can misrepresent the true dissolution rate of the dosage form. Dosage forms that float, such as capsules or floatable dosage forms, e.g. due to swelling/different technology, can yield misleading data caused by partial exposure to the dissolution media.

FIG. 1 shows a representation of the GI tract, with digestive muscular contractions, mass movement, compression, peristalsis, and other forces. All of these conditions/forces can play a key role in the rate of drug release, especially for controlled or extended release products. These mechanically destructive forces are clearly present and are imparted on a dosage form as it travels along the GI tract.

FIGS. 2 and 3 show conventional sinker devices (e.g., the Italian SkyPharma sinker and the Japanese Pharmacopoeia (JP) sinker). These sinker devices also suffer from distinct drawbacks in gathering dissolution data. When the Italian SkyPharma or the Japanese Pharmacopoeia sinker is dropped into a dissolution vessel, the dosage form may not be positioned properly at the bottom of the vessel or may be positioned in the center of the vessel perpendicular to the paddle. The design of the Italian SkyPharma and Japanese Pharmacopoeia sinkers allows the dosage form within the sinker to move or float throughout the length of the sinker. Changes in the dosage form position beyond a one-inch diameter at the center of the vessel may lead to variability in the dissolution data.

The use of Italian SkyPharma or Japanese Pharmacopoeia sinkers for dosage forms that swell may lead to variability in the dissolution data. The Italian SkyPharma or Japanese Pharmacopoeia sinkers position the dosage form such that the dosage form will swell only from the top surface. The flow of fresh dissolution media to the bottom surface of the dosage form is limited or non-existent. Italian SkyPharma or Japanese Pharmacopoeia sinkers may lead to variability in dissolution data caused by clumping of the eroded particles at the bottom of the vessel as the dosage form is dissolved or eroded. The clumped particles reduce the surface area of the dosage form that is exposed to fresh dissolution media. The longer size of the Italian SkyPharma sinkers can interfere with the paddles as they rotate, damaging the paddle or the sinkers, further leading to variability in dissolution data.

### SUMMARY OF THE INVENTION

The present disclosure provides a more accurate process and apparatus for analyzing and/or predicting release of active agents from pharmaceutical and pharmaceutical-like products.

The present disclosure also provides such a process and apparatus that more adequately replicates or simulates the conditions found in the GI tract.

The present disclosure further provides such a process and apparatus that more efficiently performs such analysis and/or predicts active agent(s) release.

There is a further need for such an apparatus and process to more adequately replicate or simulate the conditions in the GI tract.

There is yet a further need to lower variability common in dissolution data obtained with the various conventional sinkers.

These and other advantages and benefits of the present disclosure are provided by a sinker for a dissolution device to analyze the release of an active agent from a dosage form. The sinker has a first portion having an arcuate shape, a second portion having an arcuate shape, and a connector for releasably securing the first and second portions. The first and second portions define a housing for the dosage form. The first and second portions have openings therein for the flow of a dissolution medium therethrough.

In another aspect, a dissolution device to analyze the release of an active agent from a dosage form is provided, which comprises a vessel having an open end and a medium therein; a sampler that obtains a sample of the medium for analysis; and a sinker. The sinker has first and second portions with hemispherical shapes, a connector for releasably securing the first and second portions and a retainer that separates the dosage form from the first and second portions. The first and second portions define a housing for the dosage form and have openings therein for the flow of the medium therethrough.

In yet another aspect, a method of analyzing the release of an active agent from a dosage form is provided which comprises positioning the dosage form in a housing; positioning the housing in a vessel with a medium therein; flowing the medium through the housing and contacting substantially all of the dosage form with the flowing medium; and collecting data representative of the release of the active agent from the dosage form.

The sinker can have a retainer that separates the dosage form from the first and second portions of the housing. The retainer may be a wire. The retainer can also be a resilient wire that biasingly holds the dosage form. The first and second portions can be substantially equal in size. The first and second portions may also be substantially equal in size and shape.

The device can also have an impeller that circulates the medium. The device may also have a controller operably connected to the sampler that selectively obtains a sample, processes the sample, and analyzes the sample. The controller can perform UV analysis on the sample.

The method may further comprise controlling an amount of flowing of the medium. The method can also comprise resiliently holding the dosage form in the housing. The method may also comprise centering the housing along a bottom of the vessel. The sinker may also be used in conjunction with a laminar flow-through column dissolution apparatus whereby the tablet or capsule is introduced into the flowing medium encased in the sinker, thereby ensuring appropriate orientation as well as protection from any propensity to adhere to surfaces that the tablet or capsule may have. Similarly, the sinker may be used in conjunction with the reciprocating cylinder-type apparatus, also conferring a desired orientation of the dosage form to the apparatus and protecting against inappropriate adhesion to surfaces.

Other and further objects, advantages and features of the present invention will be understood by reference to the following:

### BRIEF DESCRIPTION OF THE DRAWINGS

FIG. 1 is a schematic representation view of a portion of a human upper GI tract;

FIG. 2 is a perspective view of a conventional sinker device;

FIG. 3 is a perspective view of another conventional sinker device;

FIG. 4 is a perspective view of a sinker device of the present disclosure that has a press-type connector to secure the upper and lower housing;

FIG. 5 is a cross-sectional view of the sinker device of FIG. 4, that has a twisted-tie wire connector to secure the upper and lower housing;

FIG. 6 is another perspective view of the sinker device of FIG. 4;

FIG. 7 is an exploded perspective view of the sinker device of FIG. 4;

FIG. 8 is a perspective view of the sinker device of FIG. 4 in a dissolution vessel with an impeller;

FIG. 9 represents dissolution results for enteric coated tablets over time for a dissolution apparatus using no sinker, various conventional sinkers and the sinker of the present disclosure;

FIG. 10 represents dissolution results for enteric coated tablets over time for a dissolution apparatus using no sinker, various conventional sinkers and the sinker of the present disclosure;

FIG. 11 represents dissolution results for enteric coated tablets showing % RSD (rate of dissolution) over time for a dissolution apparatus using no sinker, various conventional sinkers and the sinker of the present disclosure; and

FIG. 12 represents dissolution results for enteric tablets showing mean (% dissolved) over time for a dissolution apparatus using no sinker, various conventional sinkers and the sinker of the present disclosure.

### DETAILED DESCRIPTION OF THE INVENTION

The sinker of the present disclosure confers several advantages upon conventional paddle dissolution testing compared to other sinker devices. The sinker and accompanying vessel are suited to utilize a range of paddle speeds. The design of the sinker will prevent it from becoming clogged or allowing adhesion to the vessel sidewall, a common occurrence with matrix tablets. Eroded particles of the dosage form always gather at the bottom of the vessel and are subjected to the same level of agitation. The present sinker prevents matrix tablets from sticking to the bottom of the vessel due to an elevated platform for the tablets and capsules. The entire surface of the dosage form is uniformly exposed to the dissolution media and the orientation of the tablet/capsule can be restricted to preferably a 2.5 cm diameter of the bottom of the vessel. Discreteness of the dosage form is not destroyed as it is covered by a mesh dome. Capsules and low-density tablets will not float. Data generated using sinkers with floating capsules and other floatable dosage forms due to swelling/gas evolving technologies will be of great value for formulators in the development stage and in production. The sinker is easy to handle and to clean.

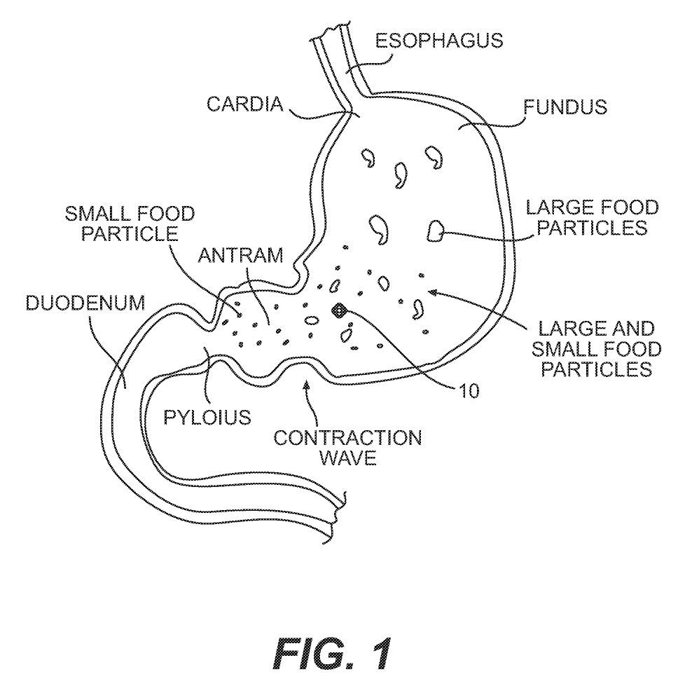

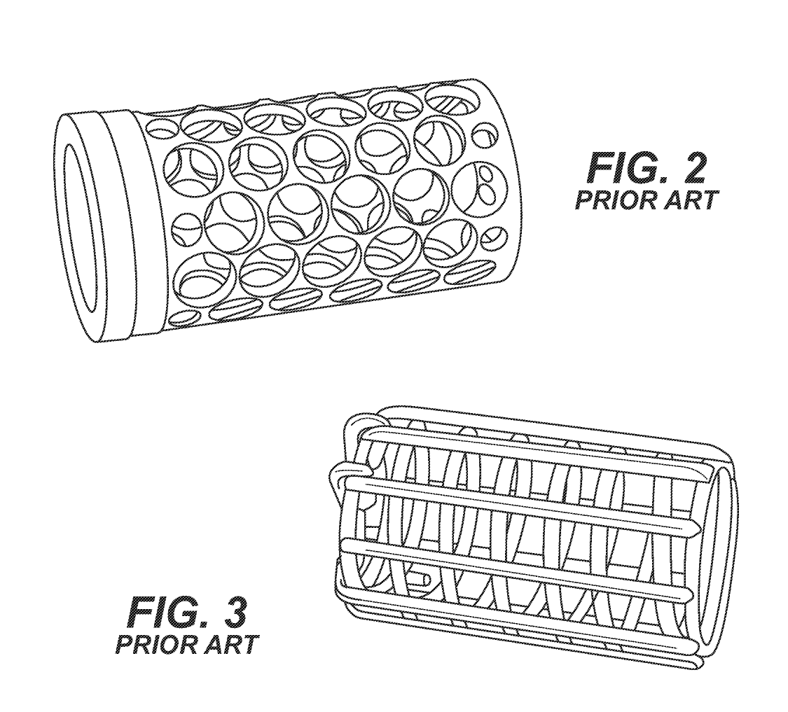

Referring to the drawings, and in particular FIG. 1, a pharmaceutical product or dosage form 10 travelling along the human GI tract is subjected to forces from a variety of sources including food and liquids that are present therein, digestive muscular contractions, mass movement, compression, peristalsis, and other forces. These forces act upon dosage form 10, effecting the release of the dosage form's active agent(s). It should be understood that while the following disclosure describes the pharmaceutical product or pharmaceutical-like product as a dosage form 10, the present disclosure contemplates analysis of any type of pharmaceutical product or pharmaceutical-like product that has an active agent(s) which is released, such as, tablets, capsules, caplets, or other dosage forms.

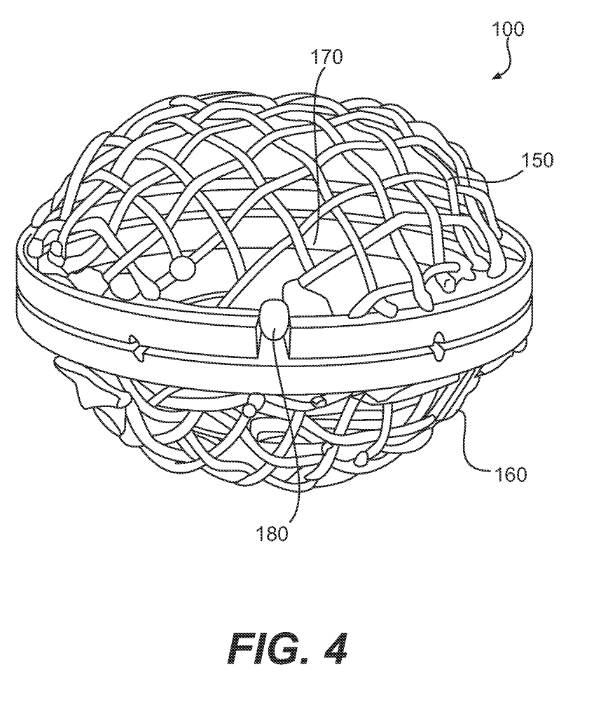

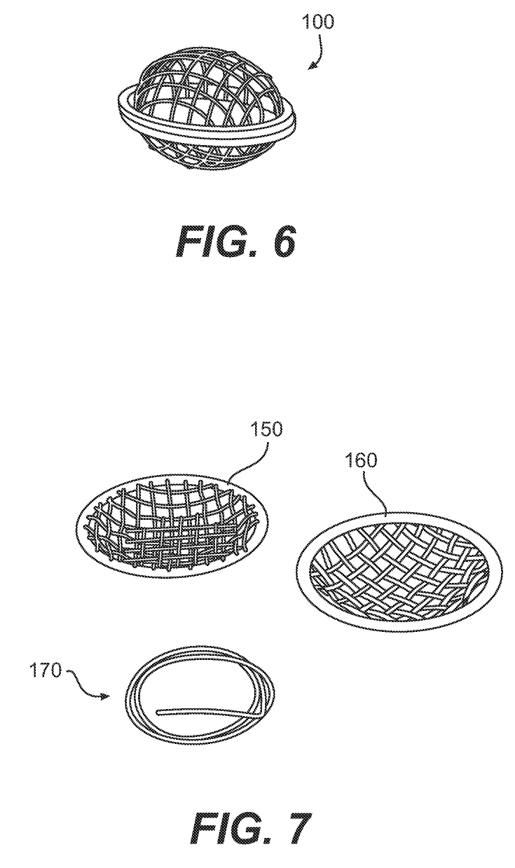

Referring to FIGS. 4 through 7, an exemplary embodiment of the pharmaceutical analysis apparatus or device of the present disclosure is shown and generally referred to by reference numeral 100. The device 100 has an upper housing 150, a lower housing 160, a platform or retainer 170 and a connector 180.

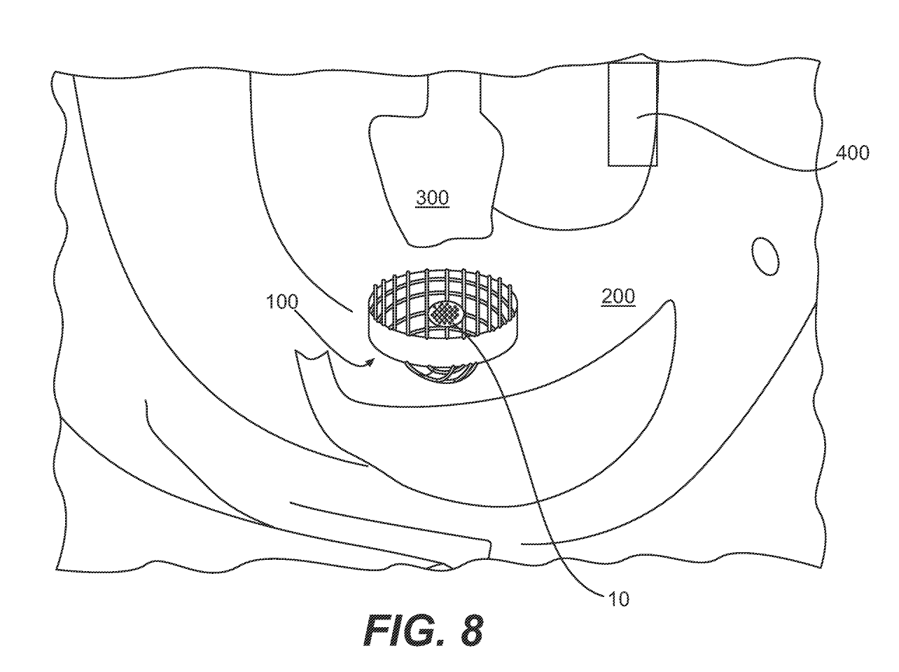

Upper and lower housings 150 and 160 have an arcuate, curved or hemi-spherical shape. The upper and lower housings are substantially equal in size and shape. This arcuate shape causes sinker 100 to move to the bottom center of the dissolution device (shown in FIG. 8). By curving both upper and lower housings 150 and 160, the orientation of sinker 100 does not prevent its ability to move to the bottom center of the dissolution device.

The upper and lower housings 150 and 160 take the form of a mesh-like structure that defines openings therein that allow the dissolution media to flow therethrough and agitate the dosage form therein. Wire meshes of various mesh sizes can be used for housings 150 and 160. The present disclosure contemplates the use of various materials for the upper and lower housings 150 and 160, such as stainless steel or plastics, including those used in the traditional USP 3 dissolution apparatus. The mesh or opening size can also be varied as appropriate for the particular dosage form 10.

The platform 170 is positioned within the upper and lower housings 150 and 160 and holds or suspends the dosage form therein. Platform 170 is a ring or wire-like structure that engages the dosage form. However, other structures and shapes can be used for platform 170. Platform 170 separates the dosage form from upper and lower housings 150 and 160 so that substantially all of the surface of the dosage form is subjected to agitation by the dissolution media. The use of a wire as platform 170 minimizes the blocked surface area of the dosage form. Additionally, the wire can be resilient and be fashioned into a ring-like form that reduces in diameter to maintain its hold on the dosage form as the dosage form decreases in size during dissolution.

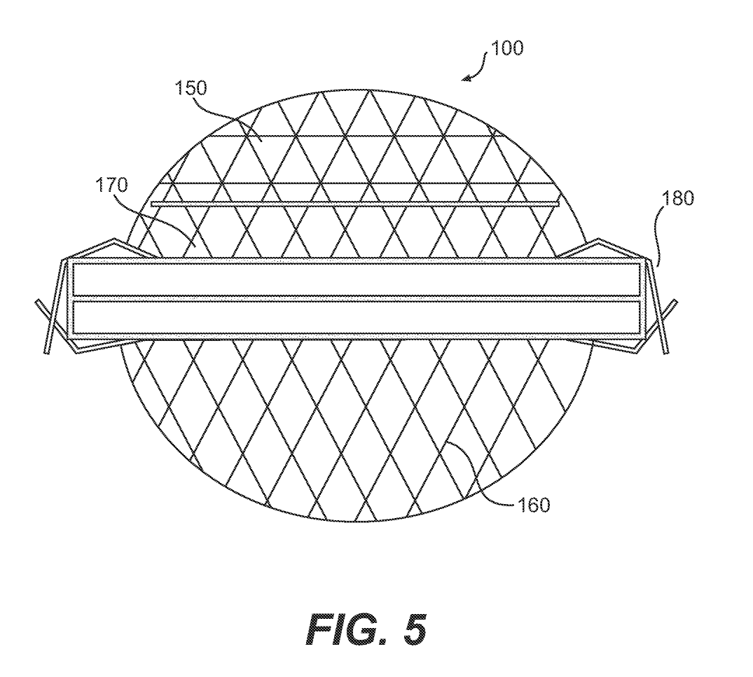

Connector 180 secures the upper and lower housings 150 and 160 but allows for easy disassembly. Connector 180 may take any of a variety of configurations so long as it secures the upper and lower housings 150 and 160 and provides for easy release. Referring to FIG. 4, the connector 180 may be in the form of a knob or press-type connector. Additional designs of connector 180, as shown in FIG. 5, may include a twisted-tie wire connector.

Referring to FIG. 8, vessel 200 holds the dissolution media, e.g., an aqueous solution, which simulates the medium in the human GI tract. The vessel is preferably a transparent, round-bottomed vessel. However, the present disclosure contemplates the use of other materials and other shapes for vessel 200, which facilitate use of sinker device 100 and/or more accurate simulation of the conditions of the GI tract.

Impeller 300 provides motion to the aqueous solution to distribute the active agent in the solution and to further simulate the conditions of the GI tract. The present disclosure contemplates the use of various shapes and sizes for impeller 300, as well as various directions of movement for the impeller (e.g., rotational and/or axial), which can facilitate distribution of the active agent in the solution and/or more accurately simulate the conditions in the GI tract. The present disclosure also contemplates the use of other devices for distributing the active agent in the solution and for simulating the motion of the medium, solution and/or dosage form 10 in the GI tract, such as, for example, a reciprocating cylinder in a cylindrical vessel.

A sampler 400 obtains samples of the aqueous solution to determine the amount of active agent that has been released by dosage form 10. Preferably, sampler 400 is operably connected to a controller, such as, for example, a control processing unit or PLC (not shown), which can selectively obtain a sample, process it, and/or analyze it. A preferred analysis is UV analysis. However, the present disclosure contemplates the use of other analytical techniques.

Sinker device 100 is preferably constructed of materials that are able to withstand prolonged exposure to acidic and to basic pH with and/or without various surfactants commonly used in pharmaceutical dissolution analysis. A preferred material is electropolished stainless steel.

The targeted types of dosage forms that will benefit more from this analysis of the release of active agents in pharmaceutical and pharmaceutical-like products are, for the most part, controlled or extended-release products. However, the present disclosure contemplates the use of this apparatus and method on all types of pharmaceutical products, including immediate release dosage forms.

It should be understood that the apparatus and method described herein has been discussed with respect to simulating the conditions in the human GI tract. However, the present disclosure contemplates the use of the apparatus and method for simulation of other GI tracts where applicable.

To demonstrate advantages of sinker 100 (labeled SeaShell sinker), dissolution testing sets of enteric coated tablets in Tris Phosphate buffer at 7.4 pH (following an initial exposure to acidic conditions) was carried out using Italian SkyPharma, Japanese Pharmacopoeia, and SeaShell sinkers 100 of the present disclosure. A semi-automated Sotax Dissolution Testing Apparatus (Model #AT7, Serial #01.3.002) was used to conduct the experiments.

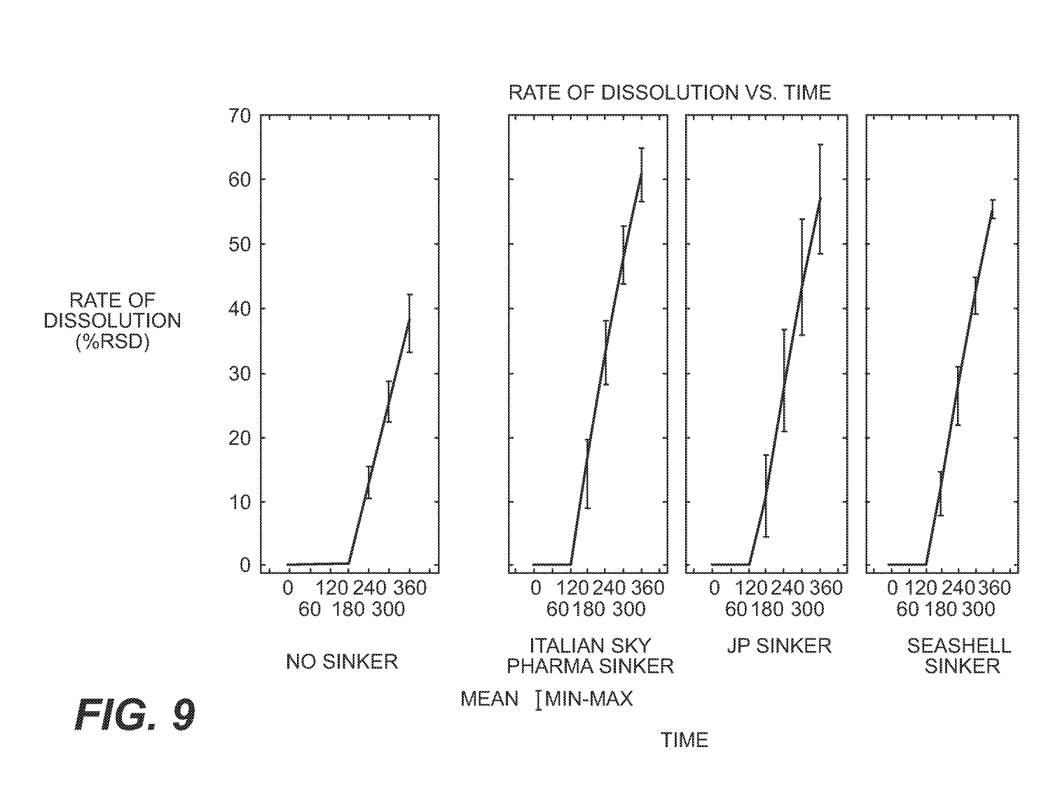

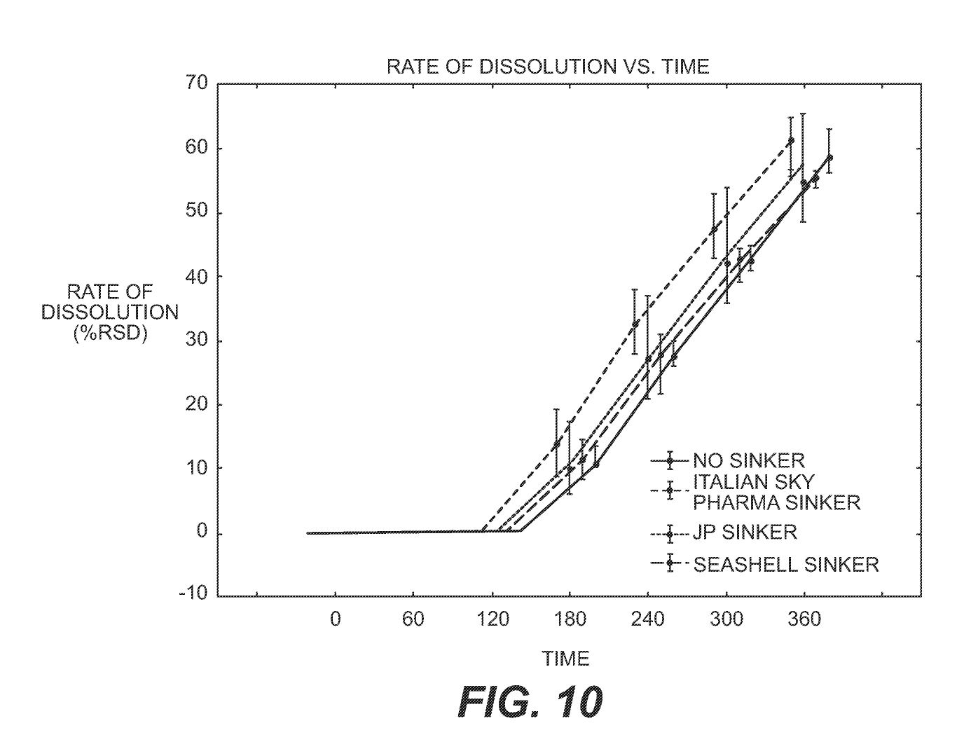

FIGS. 9 and 10 show the improved precision of sinker device 100 as compared to other conventional sinker devices and a control without a sinker device in a dissolution device for predicting dissolution of tablets. The dissolution data obtained from sinker 100 exhibited lower variability compared to the Italian SkyPharma and Japanese Pharmacopoeia sinkers.

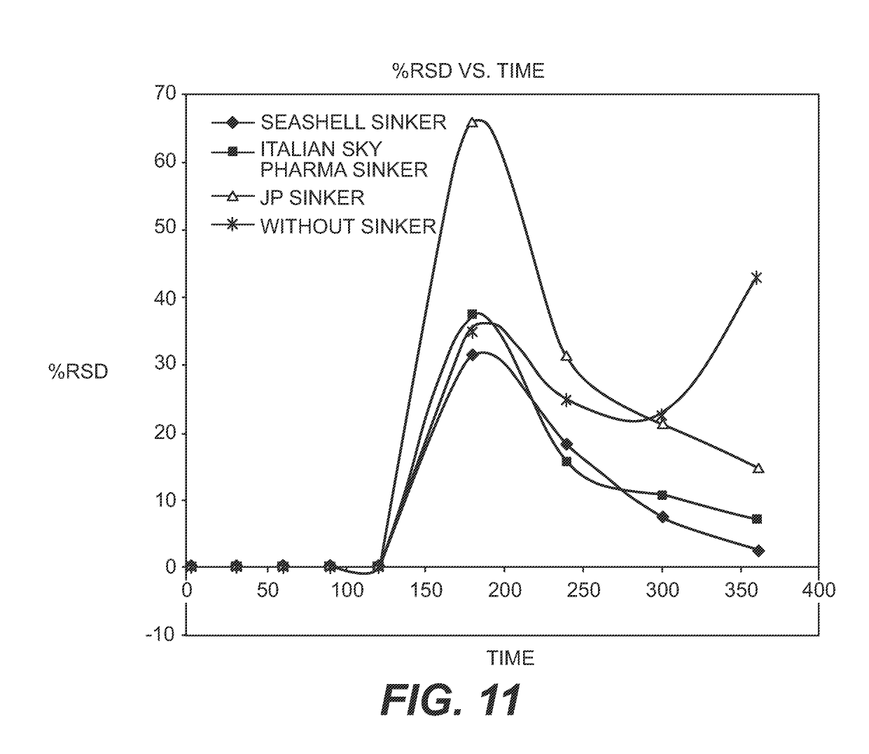

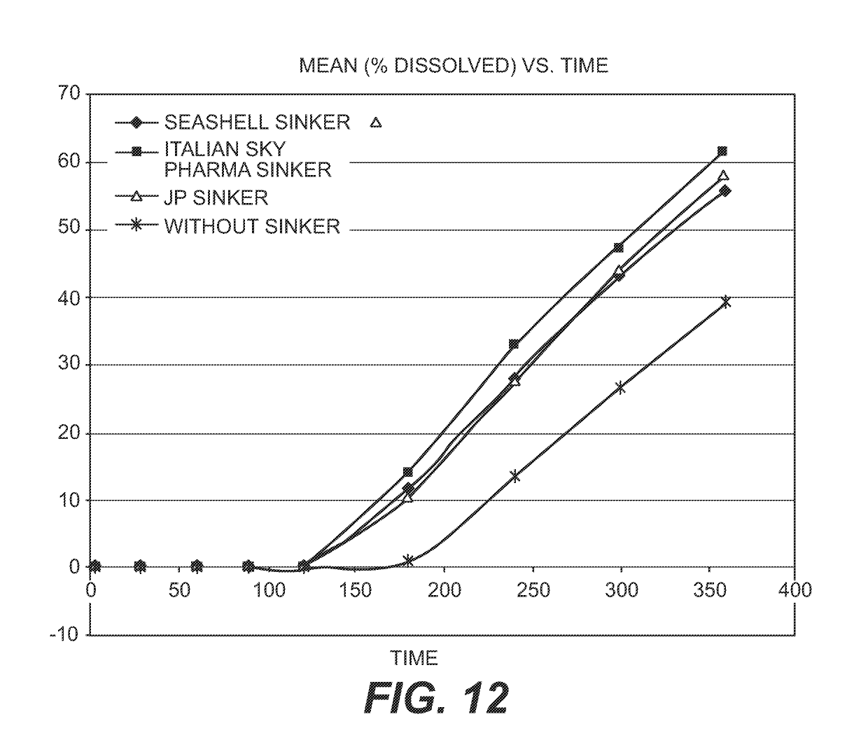

FIG. 11 shows dissolution results for tablets showing % RSD (rate of dissolution) over time for a dissolution apparatus without a sinker, various sinkers and the sinker of the present disclosure. FIG. 12 shows dissolution results for tablets showing mean (% dissolved) over time for a dissolution apparatus using no sinker, various conventional sinkers and the sinker of the present disclosure.

The shape of sinker 100 ensures that it is positioned at the center of the dissolution vessel. The variable weight distribution in sinker 100 (heavier at the bottom and lighter at the top) ensures proper orientation in the vessel. The short diameter of sinker 100 limits the movement of the dosage form. Sinker 100 allows the eroded particles to move away for the dosage form, and allows the swelling of the dosage form to occur from all directions. Sinker 100 allows the dissolution media to reach the entire surface of the dosage form at all times. Sinker 100 does not hinder swelling and allows the dosage form to stay intact.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments as described herein and in the claims.

1. A sinker for use in a dissolution device to analyze the release of an active agent from a dosage form, the sinker comprising:
a first portion having an arcuate shape;
a second portion having an arcuate shape; and
a connector for releasably securing the first and second portions, wherein the first and second portions define a housing for the dosage form, and wherein the first and second portions have openings therein for the flow of a dissolution medium therethrough.

2. The sinker of claim 1, further comprising a retainer that separates the dosage form from the first and second portions of the housing.

3. The sinker of claim 2, wherein the retainer is a wire.

4. The sinker of claim 2, wherein the retainer is a resilient wire that biasingly holds the dosage form.

5. The sinker of claim 1, wherein the first and second portions are substantially equal in size.

6. The sinker of claim 1, wherein the first and second portions are substantially equal in size and shape.

7. The sinker of claim 1, wherein the connector is selected from a group consisting of a press-type connector and a twisted-tie wire connector.

8. The sinker of claim 1, wherein the sinker has the ability to be used in a reciprocating cylinder-type dissolution apparatus.

9. The sinker of claim 1, wherein the sinker has the ability to be used in a laminar flow column dissolution apparatus.

10. A dissolution device to analyze the release of an active agent from a dosage form, the device comprising:
a vessel having an open end and containing a medium;
a sampler that obtains a sample of the medium for analysis; and
a sinker having first and second portions with hemispherical shapes, a connector for releasably securing the first and second portions and a retainer that separates the dosage form from the first and second portions, wherein the first and second portions define a housing for the dosage form and wherein the first and second portions have openings therein for the flow of the medium therethrough.

11. The device of claim 10, wherein the retainer is a wire.

12. The device of claim 10, wherein the retainer is a resilient wire that biasingly holds the dosage form.

13. The device of claim 10, wherein the first and second portions are substantially equal in size.

14. The device of claim 10, further comprising an impeller that circulates the medium.

15. The device of claim 10, further comprising a controller operably connected to the sampler that selectively obtains a sample, processes the sample, and analyzes the sample.

16. The device of claim 15, wherein the controller performs UV analysis on the sample.

17. The device of claim 15, wherein the dissolution device is a reciprocating cylinder-type dissolution apparatus.

18. The device of claim 15, wherein the dissolution device is a laminar flow column dissolution apparatus.

19. A method of analyzing the release of an active agent from a dosage form, the method comprising:
positioning the dosage form in a housing;
positioning the housing in a vessel with a medium therein;
flowing the medium through the housing and contacting substantially all of the dosage form with the flowing medium; and
collecting data representative of the release of the active agent from the dosage form.

20. The method of claim 19, further comprising controlling an amount of flowing of the medium.

21. The method of claim 19, further comprising resiliently holding the dosage form in the housing.

22. The method of claim 19, further comprising centering the housing along a bottom of the vessel.

23. The method of claim 19, further comprising performing UV analysis on a sample of the flowing medium.

24. The method of claim 19, wherein the housing comprises:
a first portion having an arcuate shape;
a second portion having an arcuate shape; and
a connector for releasably securing the first and second portions, wherein the first and second portions define the housing for the dosage, and wherein the first and second portions have openings therein for flowing the medium therethrough.
